(19) United States
(12) Patent Application Publication
(10) Pub. No.: US 2005/0163827 A1
(43) Pub. Date: Jul. 28, 2005

(54) Transdermal therapeutic delivery system

(76) Inventors: Herbert Zech, Bregenz (AT); Werner Braun, Bregenz (AT)

Correspondence Address: WILLIAM COLLARD;COLLARD & ROE, P.C., 1077 NORTHERN BOULEVARD, ROSLYN, NY, 11576, US

(21) Appl. No.: 10/832,188

(22) Filed: Apr. 26, 2004

(30) Foreign Application Priority Data
Jan. 23, 2004 (AT) 84/2004

(51) Int. Cl. A61L 15/16; A61K 9/70

(52) U.S. Cl. 424/448; 604/890.1

(57) ABSTRACT

A delivery system for the transdermal administration of active ingredients in the veterinary and human medical fields, which has a cover layer (1) and is provided with an adhesive (2) for skin adhesion, the cover layer (1) being provided with a resealable filling point (4, 8) for introducing the active ingredient through the cover layer (1). The resealable filling point (4, 8) is formed in this case by a valve (4), preferably a Luer connector (4) integrated into the cover layer (1), or by a self-sealing strip (8) made of rubber and/or mixtures of rubbers, which is integrated into the cover layer (1) or attached to the cover layer (1).

The present invention relates to a transdermal therapeutic delivery system according to the preamble of Claim 1.

Transdermal therapeutic delivery systems of this type, also referred to in the following as TTS for “transdermal therapeutic system”, have been known for some time and have the goal of administering therapeutically active substances via the skin. The form and construction of these TTS vary, however they generally include a cover layer and possibly further layers which either define a cavity as a reservoir for the active ingredient or contain a matrix in which the active ingredient is embedded. An adhesive film ensures the adhesion on the skin of the patient. In this case, the active ingredient first diffuses through the uppermost skin layer (stratum corneum) and finally reaches lower-lying layers of the skin, where the active ingredient is absorbed by blood capillaries and thus distributed in the body of the patient via the cardiovascular system. A known example of such a TTS is the nicotine patch for smoking withdrawal, however, in the meantime multiple other active ingredients may be administered with the aid of TTS, such as fentanyl, nitroglycerin, estradiol, ethinyl estradiol, norethindrone acetate, testosterone, clonidine, lidocaine, prilocaine, or scopolamine.

Principally, one differentiates between active and passive types of administration in regard to TTS. The passive type of administration is essentially based on a diffusion of the active ingredient into the skin which is caused by a concentration gradient, i.e., the difference between the high active ingredient concentration existing in the TTS and the low concentration existing in the skin. The applicability of this method requires suitable physical-chemical properties of the active ingredient, particularly in regard to the molecular size of the active ingredient. Generally, it is thought that only active ingredients up to a molecular size of approximately 1000 Dalton are suitable for such a passive type of administration, which clearly limits the number of active ingredients usable for TTS.

The usability of TTS was decisively expanded by the development of active types of administration. The diffusion-controlled transport of the active ingredient into the deeper skin layers is improved hereby through different measures, for example, additional force gradients are provided that support its transport. An example of an active type of administration is phonophoresis (or also sonophoresis), for example, in which ultrasound waves are used in order to expand the intercellular space and allow the penetration of larger molecules, iontophoresis, in which electrical currents of low intensity are applied in order to transport polar or ironic molecules into the deeper skin layers through electrical repulsion, or electroporation, in which pulsed electrical fields are applied that cause a temporary elevation of the permeability of cell membranes by forming “microchannels”. Furthermore, types of administration using thermal energy are also known, which also improve the permeability of the skin, or using microneedles, which penetrate the uppermost skin layers in a mechanical way without irritating nerve endings lying underneath and are either coated with the active substance to be administered or are provided with cannulas for transporting the active ingredient. With the aid of active systems of this type, the methods of transdermal administration may be expanded to molecules with sizes up to 20,000 Dalton, so that a range of peptides, proteins, and hydrocarbons which is of great therapeutic interest becomes accessible.

Active and passive types of administration may also be combined. Thus, for example, elevating the permeability of the skin with the aid of electroporation before sticking on a passive TTS, for example, and only then sticking on a TTS is known. In this case, not only is the ability to transport larger molecules into the deeper skin layers provided, but rather also the ability to elevate the. diffusion rates and therefore the dose capacity of the administration, which is decisive for many applications. With the aid of methods of this type, it became possible to apply active ingredients of different fields, such as pain therapy, the treatment of neurological ailments, cardiovascular diseases, respiratory diseases, or diseases of the locomotor apparatus, such as osteoporosis, with the aid of TTS. Insulin and calcitonin are particularly prominent examples of active ingredients which may be administrated using TTS in the meantime.

The applicability of TTS is, however, subjected to a strong restriction in that the active ingredient is to be stored in liquid solution or as a suspension in the reservoir or the matrix of the TTS. However, many active ingredients are unstable in solution or in suspension, so that TTS for these active ingredients would only have a low storability, which makes their applicability impractical. Active ingredients of this type are administered subcutaneously or intramuscularly using injection needles, as before.

Many of these active ingredients must, however, be administered regularly, in some circumstances even daily. Therefore, the requirement for a daily doctor visit arises for the patient, or he administers the injections himself. Examples of these are human gonadotropins (FSH/LH, hMG, hCG) or recombinant human gonadotropins, recombinant human follicle-stimulating hormone (rFSH), recombinant human luteinizing hormone (rLH), or GnRH agonists and/or GnRH antagonists, which are to be administered in the course of in vitro fertilization treatments for encouraging ovulation and/or egg cell maturation. Active ingredients of this type are distributed in the pharmaceutical market as powders, for example, which are dissolved directly before the injection in water, or as liquid solutions which are to be cooled continuously.

However, the independent application of injections is not only unpleasant, but rather also cannot be expected of everyone, in particular, it is forbidden in many countries, such as Japan, for women to administer injections to themselves.

It is therefore the object of the present invention, with the aid of a correspondingly constructed transdermal delivery system, to provide a possibility for administering active ingredients which are currently not suitable for typical transdermal delivery systems without the aid of injection syringes. The delivery system is to be suitable in this case for multiple active ingredients without having to change the delivery system decisively.

This object is achieved by the characterizing features of Claim 1.

Claim 1 hereby provides that the cover layer of the delivery system is provided with a resealable filling point for introducing the active ingredient through the cover layer. The necessity for already equipping the delivery system with the active ingredient by the manufacturer is therefore dispensed with. Rather, it may be acquired in the market as a delivery system without active ingredient which is only “filled” with the active ingredient by the patient directly before the application. In this case, the active ingredient is distributed in the typical way in cooled, liquid form or in powdered form, the powder, as also typical until now in the framework of self injection, being dissolved by patients in a suitable concentration and quantity.

The resealable filling point may be implemented in different ways. Thus, for example, Claim 2 provides that the resealable filling point is formed by a self-sealing strip made of rubber or mixtures of rubbers which is integrated into the cover layer or attached to the cover layer. If this strip is punctured with the aid of an injection needle, for example, in order to introduce active ingredient under the cover layer, the resulting fine opening is sealed again after pulling out the needle because of the self-sealing properties of the rubber material. This effect is currently used in the medical field for injection and infusion bottles, for example.

Another possibility is suggested in Claim 3, according to which the resealable filling point is formed by a valve, preferably a Luer connector, integrated into the cover layer. In the framework of this embodiment, the use of injection needles may be dispensed with completely, since only the corresponding counterpart for producing a Luer Lock seal is to be attached.

Particularly if rubber strips are used as the filling point, it is advantageous according to Claim 4, for better ability to fill the delivery system, for spacer webs to be located on the resealable filling point on the side of the cover layer facing toward the skin surface, to ensure a defined spacing position of the cover layer to the skin surface after sticking on the delivery system. Furthermore, according to Claim 5, the spacer webs may be covered on their side facing toward the skin surface with a protective lamina essentially parallel to the cover layer. In this way, puncturing the skin surface may be avoided, during the introduction of an injection needle into the delivery system, for example.

Although it is primarily only necessary to provide a cover layer and an adhesive for skin adhesion to implement the delivery system according to the present invention, an additional semipermeable membrane may also be used according to Claim 6, which is attached on the side of the cover layer facing toward the skin surface, at least one cavity being defined between the cover layer and the membrane. Membranes of this type are also used in known transdermal delivery systems and are particularly used for the purpose of controlling the delivery rate of the active ingredient to the lower skin layers. According to Claim 7, the cavity has a partial vacuum in comparison to the surrounding atmosphere. In this way, upon application of an injection needle and/or upon production of a Luer Lock seal, the active ingredient is suctioned from the external depot into the cavity.

Claims 8 and 9 provide preferred materials for the cover layer and/or for the membrane. Claims 10 through 15 finally suggest concrete possible applications of the delivery system according to the present invention for specific active ingredients.

The present invention will now be described in greater detail in the following on the basis of the attached figures. In this case,

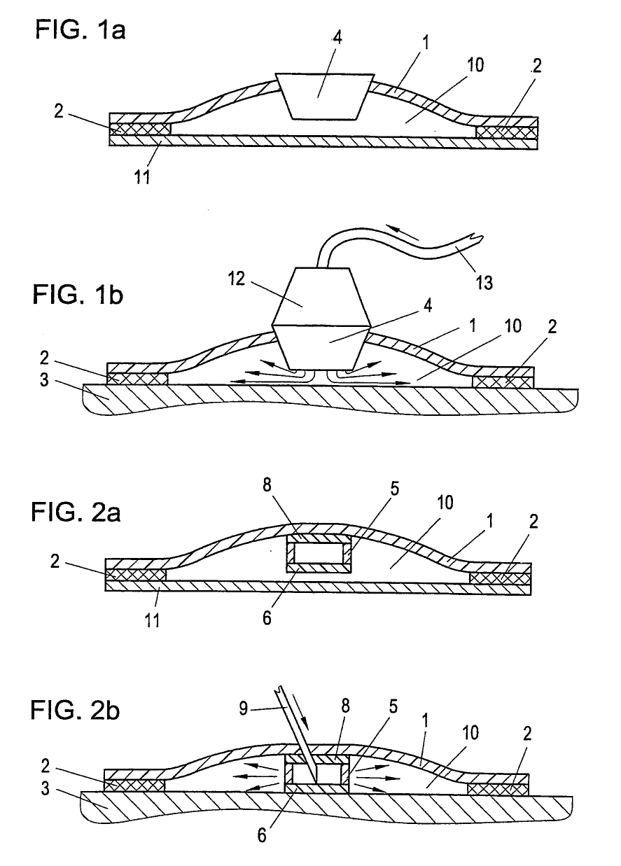

FIG. 1a shows a schematic illustration of an embodiment of a delivery system according to the present invention using a valve,

FIG. 1b shows the embodiment shown in FIG. 1a after being stuck on the skin surface,

FIG. 2a shows a schematic illustration of a further embodiment of a delivery system according to the present invention using a rubber strip,

FIG. 2b shows the embodiment shown in FIG. 2a after being stuck on the skin surface,

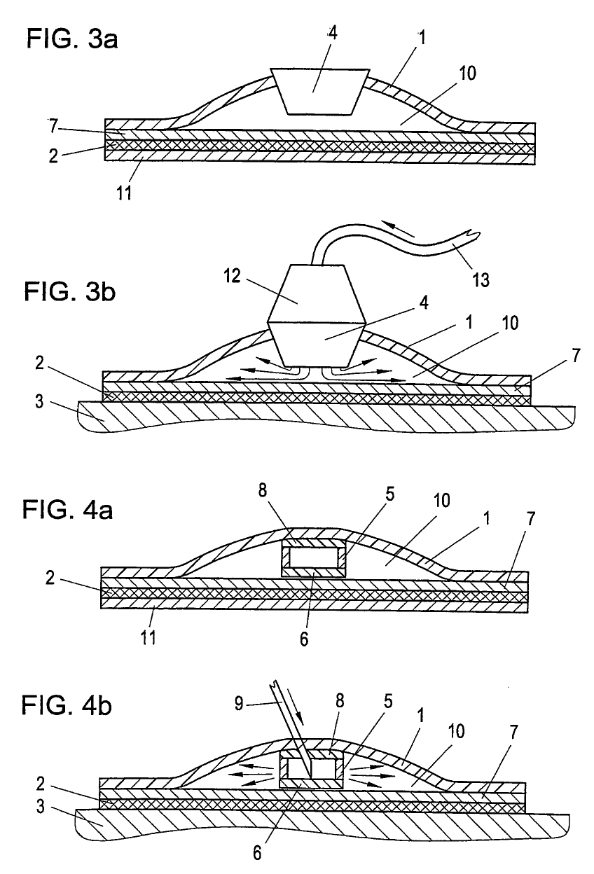

FIG. 3a shows a schematic illustration of a further embodiment of a delivery system according to the present invention using a valve and an additional membrane,

FIG. 3b shows the embodiment shown in FIG. 3a after being stuck on the skin surface,

FIG. 4a shows a schematic illustration of a further embodiment of a delivery system according to the present invention using a rubber strip and an additional membrane, and

FIG. 4b shows the embodiment shown in FIG. 4a after being stuck on the skin surface.

FIG. 1a shows a schematic illustration of an embodiment of a delivery system according to the present invention having a cover layer 1, in which a valve 4, preferably a Luer connector 4, is integrated. The cover layer 1 has the object of protecting the active ingredient introduced and holding it in the application region. At the same time, it is to have the lowest possible permeability to moisture. This prevents the active ingredient, which is typically provided in liquid solution and/or suspension, from escaping and also avoids drying of the skin surface 3 at the contact points with the cover layer 1. Furthermore, it is to have good permeability for oxygen, so that the skin respiration is not suppressed at these contact points. In addition, the cover layer 1 is to be elastic enough in order to adapt its shape to the application region. Therefore, polyethylene is a suitable material for this cover layer 1, for example. Furthermore, laminates based on polyester are also conceivable, in which a polyethylene layer is combined with a polyester layer, for example. Such structures having a multilayered construction are also included in the following by the reference “cover layer 1”. For many applications, the inclusion of vinyl acetate may also prove itself, in order to improve the chemical compatibility with many active ingredients or also the ability to bond with the membrane 7. Furthermore, metal films, such as aluminum film, may also be laminated with the polymer cover layer 1 in order to provide the delivery system with additional strength. Depending on the application, multilayered cover layers 1 may therefore be constructed, for example, that have a laminate made of polyethylene of moderate density and a polyester-polythylene terephthalate, onto which a thin layer of aluminum is vapor deposited. Furthermore, polymers treated with silicone may be used, such as polyalkylene terephthalate, alone or in a laminate. The ability of the different layers to bind may be improved with the aid of binding agents such as polyurethanes or ionomers in this case. Techniques of this type for producing multilayered structures from polymer materials are sufficiently known to those skilled in the art.

As is also visible from FIG. 1a , according to one embodiment, a valve 4, preferably a Luer connector 4, is integrated into the cover layer 1. In this case, it may be the male or the female part of a Luer seal. Furthermore, the Luer seal may be lockable or even only able to be plugged in. Different forms of Luer closures are also well known, and their integration into films made of polymer materials represents no problem in regard to plastic technology for those skilled in the art.

As may also be seen from FIG. 1a, the cover layer 1 is provided with layer made of an adhesive 2 which ensures the adhesion of the TTS on the skin surface. Silicone, acrylate, or polyisobutylene (PIB) adhesives have proven suitable as the adhesive 2, furthermore, cross-linked copolymers of dimethyl amino ethyl methacrylate and an alkyl acrylate or mixtures of 2-cyanoacrylate and dimethyl methylene malonate may also be used. Further examples are esters of α-cyanoacrylic acid, adhesives based on a hydrocolloidal rubber, silicone adhesives for the medical field, or cross-linked dextran. The adhesive 2 will only extend over the edge regions of the cover layer 1 for TTS which only include the cover layer 1, in order to ensure the implementation of a cavity 10 after application of the TTS on the skin surface 3. If necessary, the adhesive 2 may also contain solvents in order to provide it with thixotropic properties, elevate its cohesiveness, and make it easily removable from the skin surface 3. For TTS which provide a membrane 7 in addition to the cover layer 1, the adhesive film 2 may even extend over the entire area of the membrane 7, as will be described in greater detail.

FIG. 1a shows the delivery system according to the present invention in a form as it exists before it being stuck on the skin surface 3 and is distinguished in that the cover layer 1 has a protective layer 11 on its side intended for the skin surface 3. This protective layer 11 is to ensure the sterility of the cavity 10. In this case, it must be easily detachable from the cover layer 1 and/or membrane 7 provided with the adhesive film 2 and may not remove the adhesive film 2 as it is detached. Protective layers 11 based on fluoropolymer and/or fluoropolymer-coated polyester layers have been shown to be suitable for this purpose, however, they may also be manufactured from any of the materials which were also cited for cover layer 1, if they may be pulled off. Further examples of known materials which may be used for the production of the cover layer 11 include polytetrafluoroethylene, cellophane, paper treated with silicone, films made of polyvinylchloride, or even polyester treated with silicone.

As may be seen from FIG. 2a , the filling point may also be implemented with the aid of a strip 8 made of rubber or a mixture of rubbers. These are understood here to include natural rubber, balata, gutta-percha, guayule, chicle, and similar natural types of rubber, as well as synthetic rubber, factice, and their regenerates. Butyl, chlorobutyl, natural, EPDM, or silicone rubber may also be cited as concrete examples. In the field of pharmaceutical rubber seals, rubber types such as isoprene, ethylene propylene, butadiene, or even liquid or solid silicone are known in particular. It is only decisive for the delivery system according to the present invention that it is to have self-sealing properties, i.e., it is to seal itself again after puncture with a thin cannula. The production of rubber strips having these properties is well known in this case. The attachment on the cover layer 1 may be performed using gluing, for example, the attachment able to be performed on the outside of the cover layer 1 or on the inside 1. For reasons of sterility, however, it is preferable to attach it on the inside of the cover layer 1, i.e., inside the cavity 10. The shaping of the rubber strip 8 may vary, thus, it may be implemented as approximately circular or rectangular. The size of the rubber strip may also be selected freely, however, it is to be large enough in order to be easily found and punctured by the patient. For this purpose it is advantageous, for example, to make the region of the cover layer 1 on which the rubber strip 8 is attached a different color than the remaining regions of the cover layer 1, in order to make finding the filling point easier. It would also be conceivable, however, to have the self-sealing rubber strip 8 extend over the entire cover layer 1, so that it represents an additional layer of the TTS, as long as the requirements for the TTS, particularly in regard to elasticity and flexibility, remain fulfilled.

Furthermore, it may be seen from FIG. 2a that according to a preferred embodiment, spacer webs 5 are located on the resealable filling point 8 on the side of the cover layer 1 intended for the skin surface 3 to ensure a defined spacing position of the cover layer 1 from the skin surface 3 after the delivery system is stuck on. These spacer webs 5 ensure better ability to fill the delivery system by supporting the rubber strip 8 from the skin surface 3 as it is punctured by the cannula 9, for example. They may be manufactured from hard PVC, for example, and either compressed with the rubber strip 8 or glued thereon. The spacer webs 5 may also be manufactured integrally with the cover layer 1, the rubber strip 8 being pressed or glued between the spacer webs 5. The shaping of these spacer webs 5 may vary, for example, they may be positioned circularly or rectangularly around the rubber strip 8. It is essential that they define intermediate spaces, through which the active ingredient may reach the cavity 10 upon injection, as is indicated in FIG. 2b by the arrows. Furthermore, the spacer webs 5 may be covered on their side intended for the skin surface 3 by a protective lamina 6 which is essentially parallel to the cover layer 1. In this way, upon introduction of an injection needle 9 (FIG. 2b ) into the delivery system, for example, pricking of the skin surface 3 may be avoided. The protective lamina 6 may also be manufactured from hard PVC, for example, and glued or shaped onto the spacer webs 5. The shaping of the protective lamina 6 may also vary, thus, it may be implemented as circular or rectangular, for example. It is again essential in this case that the spacer webs 5 and the protective lamina 6 define intermediate spaces which allow the active ingredient introduced to enter the cavity 10.

It is to be noted that the individual components of the delivery system according to the present invention in FIGS. 1 through 4 are not shown in scale to one another so they may be illustrated more easily. Thus, for example, the cover layer 1 or the adhesive layer 2 will be thinner in practice in relation to the rubber strip 8 or the spacer webs 5 than shown in FIGS. 1 through 4.

For the embodiments shown in FIGS. 1a and 2a , a membrane 7 may additionally be provided, as is shown in FIGS. 3a and 4a . The membrane 7 represents a structural reinforcement and also ensures a controlled diffusion of the active ingredient to the skin surface 3. Since a diffusion in the direction of the active ingredient reservoir is to be suppressed, semipermeable membranes 7 are used. For this purpose, ethylene-vinyl acetate membranes or other copolymers made of ethylene-vinyl methyl acetate, ethylene-vinyl ethyl acetate, or ethylene-vinyl propyl acetate have been shown to be suitable. By elevating the vinyl acetate component, the permeability and the moisture permeability may be increased in this case. Further examples of suitable materials include polyolefins such as polyethylene and polypropylene, polytetramethylene-ether terephthalate, polyisoprene, polyacrylonitrile, or ethylene-propylene copolymers, for example. If a membrane 7 is used, the adhesive 2 is obviously located on the side of the membrane 7 intended for the skin surface 3, the membrane 7 again being provided with a protective layer 11 on the side which is brought into contact with the skin surface 3, in order to ensure sterility.

FIGS. 1a, 2a , 3a , and 4a show different embodiments of the delivery system according to the present invention in a form as it exists before being stuck on the skin surface 3 and are distinguished in that the cover layer 1 has a protective layer 11 on its side intended for the skin surface 3. In contrast, FIGS. 1b, 2b , 3b , and 4b show the particular embodiments after sticking the delivery system on the skin surface 3, the protective layer 11 first having been removed from the delivery system. The delivery system may then be pressed onto a desired application region, the adhesive 2 ensuring adhesion on the skin surface 3. As indicated in FIGS. 2b and 4b , the filling point 8 may now be punctured using the cannula 9 of an injection needle, the protective lamina 6 preventing further penetration of the needle 9. In the embodiment shown in FIGS. 1b and 3b , in contrast, the counterpart 12 of the Luer seal may be attached to the Luer connector 4 after sticking on the delivery system. In the embodiments shown in FIGS. 3a and 4a , in which an additional membrane 7 is provided, the cavity 10 located between the cover layer 1 and the membrane 7 may be under partial vacuum in comparison to the surrounding atmosphere, so that after the filling point 8 is punctured and/or after the production of the Luer seal with the aid of the valve 4, the active ingredient is suctioned out of the injection needle 9 and/or an external depot (not shown in FIGS. 1 through 4) and into the cavity 10 along the directions indicated in FIGS. 1b through 4b by the arrows. In the embodiments shown in FIGS. 1a and 2a , in which no additional membrane 7 is provided, the air located under the cover layer 1 after placement on the skin surface 3 may be pressed out by smoothing the delivery system, in order to thus make the introduction of the active ingredient easier. As is indicated in FIGS. 1b and 3b with the aid of tubing 13, the active ingredient may be supplied from the external depot to the counterpart 12 of the Luer seal and finally to the Luer connector 4. Of course, the external active ingredient depot may also be placed directly on the counterpart 12. In this case, the dosing of the active ingredient is not fixed by the volume of the cavity 10, but rather is already performed by the patient even before introducing the active ingredient with the aid of a preparation of a suitable quantity, as is already typical in the current way for self injection.

In the embodiment shown in FIGS. 2a and/or 4a , after the cannula 9 is pulled out, because of the self-sealing properties of the rubber strip 8, the puncture opening is sealed again, so that the active ingredient was securely introduced into the cavity 10. In the embodiment shown in FIGS. 1a and/or 3a , the secure storage of the active ingredient in the cavity 10 is caused because of the sealing effect of the valve 4 after removal of the counterpart 12.

The shape and size of the delivery system according to the present invention may vary and will be oriented to the therapeutic requirements, i.e., to the quantity of the active ingredient to be administered or to the application region, for example. Furthermore, it may be implemented as circular or rectangular and additionally also have further imprints or colored designs for possible special identifications of the filling points, for example, to identify the top and bottom of the TTS.

In the event of administration of active ingredients having a larger molecular size, it may be shown to be advantageous or even necessary to appropriately elevate the permeability of the relevant skin surface 3 before the application of the delivery system according to the present invention with the aid of methods as were described above under the concept “vactive types of administration”. Thus, for example, before sticking on the delivery system, phonophoresis, iontophoresis, or electroporation may be performed to form microchannels. Furthermore, a prior application of thermal energy may be performed, which also improves the permeability of the skin. Through prior measures of this type, even active ingredients having relatively large molecular size, such as human gonadotropins (FSH/LH, hMG, hCG) or recombinant human gonadotropins, recombinant human follicle-stimulating hormone (rFSH), recombinant human luteinizing hormone (rLH), recombinant human choriogonadotropin (rhCG), or GnRH agonists and/or GnRH antagonists may be administered with the aid of the delivery system according to the present invention. These active ingredients are particularly also of interest in the framework of the present invention because they are sometimes to be administered daily in the course of in vitro fertilization treatments for encouraging ovulation and/or egg cell maturation. Active ingredients of this type are purchased in the typical way in the pharmaceutical market as powders, for example, which are dissolved directly before the injection in water, or they are already on the market in liquid form. The liquid solution may subsequently be introduced into the cavity 10 as described, from which it gradually diffuses through the skin surface 3. Other active ingredients are offered in liquid form, but require continuous cooling. The delivery system according to the present invention has also shown to be advantageous in this case, since only the active ingredient, but not the TTS itself, must be cooled. The TTS according to the present invention may be produced and distributed without the necessity of cooling, the patient only introducing the cooled active ingredient solution into the TTS directly before the application.

With the aid of the delivery system according to the present invention, even active ingredients which are currently not suitable for typical transdermal delivery systems are therefore also available for administration with the aid of transdermal delivery systems. In this case, the aid of injection syringes may also be dispensed with. The delivery system is suitable for multiple active ingredients in this case, without having to change the delivery system decisively.

1. A delivery system for the transdermal administration of active ingredients in the veterinary and human medical fields, which has a cover layer (1) and is provided with an adhesive (2) for skin adhesion,
wherein the cover layer (1) is provided with a resealable filling point (4, 8) for introducing the active ingredient through the cover layer (1).

2. The delivery system according to claim 1,
wherein the resealable filling point (4, 8) is formed by a self-sealing strip (8) made of rubber or mixtures of rubbers which is integrated into the cover layer (1) or attached to the cover layer (1).

3. The delivery system according to claim 1,
wherein the resealable filling point (4, 8) is formed by a valve (4), preferably a Luer connector (4), integrated into the cover layer (1).

4. The delivery system according to claim 1,
wherein spacer webs (5) are located on the resealable filling point (4, 8) on the side of the cover layer (1) facing toward the skin surface (3) to ensure a defined spacing distance of the cover layer (1) to the skin surface (3) after sticking on the delivery system.

5. The delivery system according to claim 4,
wherein the spacer webs (5) are covered with a protective lamina (6), which is essentially parallel to the cover layer (1), on their side facing toward the skin surface (3).

6. The delivery system according to claim 1,
wherein the cover layer (1) is additionally attached to a semipermeable membrane (7) on its side facing toward the skin surface (3), at least one cavity (10) being defined between the cover layer (1) and the membrane (7).

7. The delivery system according to claim 6,
wherein the cavity (10) has a partial vacuum in comparison to the surrounding atmosphere.

8. The delivery system according to claim 1,
wherein the cover layer (1) is a film made of a material selected from the group including polyethylene, laminates of polyethylene and polyester, laminates of polyethylene and a polyester-polyethylene terephthalate, polymers treated with silicone, such as polyalkylene terephthalate treated with silicone, or combinations thereof.

9. The delivery system according to claim 1,
wherein the membrane (7) is a film made of a material selected from the group including polyolefins, polytetramethylene-ether terephthalate, polyisoprene, polyacrylonitrile, ethylene-propylene copolymers, ethylene-vinyl acetate copolymers, or other copolymers made of ethylene-vinyl methyl acetate, ethylene-vinyl ethyl acetate, ethylene-vinyl propyl acetate, or combinations thereof.

10. A combination of a delivery system according to claim 1, with human gonadotropins (FSH/LH, hMG, hCG) as the active ingredient.

11. A combination of a delivery system according to claim 1, with recombinant human follicle-stimulating hormone (rFSH) as the active ingredient.

12. A combination of a delivery system according to claim 1, with recombinant human luteinizing hormone (rLH) as the active ingredient.

13. A combination of a delivery system according to claim 1, with recombinant human choriogonadotropin (rhCG) as the active ingredient.

14. A combination of a delivery system according to claim 1, with GnRH agonists or GnRH antagonists as the active ingredient.

15. A combination of a delivery system according to claim 1, with recombinant human gonadotropins as the active ingredient.
